(19) United States
(12) Patent Application Publication
(10) Pub. No.: US 2024/0185950 A1
(43) Pub. Date: Jun. 6, 2024

(54) DIGITAL ANALYSIS OF NUCLEIC ACID MODIFICATION

(71) Applicant: Bio-Rad Laboratories, Inc., Hercules, CA (US)

(72) Inventors: Michael Samuels, Windham, NH (US); Jeffrey Olson, Chelmsford, MA (US); Darren R. Link, Lexington, MA (US)

(21) Appl. No.: 18/538,076

(22) Filed: Dec. 13, 2023

Related U.S. Application Data
(63) Continuation of application No. 14/505,974, filed on Oct. 3, 2014, now Pat. No. 11,901,041.
(60) Provisional application No. 61/887,103, filed on Oct. 4, 2013.

(51) Int. Cl. G16B 20/10; G16B 20/00

(52) U.S. Cl. CPC G16B 20/10; G16B 20/00

(57) ABSTRACT

In certain aspects, methods of the invention involve performing modification state specific enzymatic reaction of nucleic acid in a sample, determining a value associated with efficiency of the modification state specific enzymatic reaction based on a control, determining an amount of target nucleic acid in the sample, and normalizing the amount of target nucleic acid based on the efficiency value. Based on the normalized amount of target nucleic acid, the method further includes determining whether the normalized amount of target nucleic acid is indicative of a condition.

### TECHNICAL FIELD

This invention generally relates to quantification of nucleic acid molecules present in a modification state using target/substrate state-dependent enzymes. For example, in one embodiment the invention comprises quantification of nucleic acid comprising a particular methylation state using methylation-specific restriction enzymes.

### BACKGROUND

It is generally understood that in biological systems nucleic acid molecules may be selectively modified in various state or condition specific ways and are thus useful as biomarkers for the various states or conditions. One well known example of nucleic acid modification includes DNA methylation which is generally appreciated as an important regulator of gene expression. DNA methylation may play a role in embryogenesis and epigenetic regulation that has been implicated in the development and progression of a number of diseases, such as cancer and osteoarthritis. For example, nucleic acid methylation in eukaryotic organisms can occur with cytosine species located 5′ to a guanine species (i.e., CpG sequences), however other forms of methylation are also known. Research suggests that genes with high levels of methylation in a promoter region are transcriptionally silent, which may allow unchecked cell proliferation. When a promoter region has significant methylation of nucleic acid bases in certain regions, the methylation is typically most prevalent in sequences having CpG repeats, so called “CpG islands.” Methylated states have also been implicated in the development and progression of cancer through different mechanisms. Continuing with the present example, other types of methylation mechanisms have also been identified and may occur in heterogeneous/mixed samples such as CpA methylation profiles associated with stem cells or hydroxymethylation.

Species of target/substrate state-dependent enzymes can be useful tools to selectively enrich for a population of nucleic acid in a certain modification state. Continuing with the example of methylation, species of methylation-specific restriction enzymes (also referred to as MSRE) provide a mechanism for enrichment of a population of nucleic acid molecules by utilizing the characteristics of one or more MSRE species that includes an inability to cleave nucleic acid molecules at the enzyme recognition site when in a particular methylation state. For example, in some embodiments when a recognition site for a certain species of restriction enzyme is methylated, the MSRE cleaves the nucleic acid, and consequently the nucleic acid molecule is enzymatically reacted. Alternatively, when the recognition site is in an un-methylated state, the species of MSRE cannot enzymatically react with the nucleic acid molecule, and the integrity of the nucleic acid molecule is preserved.

There are a number of applications where accurate quantification of differences between nucleic acid modification states is desirable in order to accurately identify a disease state or condition. That identification can be particularly challenging in cases where there are only small quantities of starting nucleic acid molecule in a sample available. An example of such a field of application includes a non-invasive test for fetal genomic abnormalities. It is highly desirable that such a test accurately quantifies the amount fetal DNA in maternal blood in order to perform aneuploidy analysis (e.g. trisomy of chromosome 21, 18, or 13). It is therefore advantageous to utilize differences in a methylation state of one or more target regions that exist between a maternal nucleic acid population and a fetal nucleic acid population to quantify the amount of fetal DNA in maternal blood to identify whether a genomic abnormality is present in the fetus. In other words, accurate determination of the percentage of fetal DNA present in a sample drawn from the mother provides confidence in the counting and allocation of chromosome number contributed from the fetal and maternal sources. Another example of a field of application includes cancer analysis where it is generally appreciated that there are differences in methylation state of certain gene targets between cancer cells and normal cells in a tissue type.

### SUMMARY

This invention generally relates to increasing the sensitivity of assays for quantification of modified nucleic acid populations using target/substrate state-dependent enzymes. In particular aspects, the invention relates to digital analysis for detection and quantification of differences between methylation-dependent targets (e.g. target regions on a nucleic acid that are in a certain methylation state). For example, methods of the invention utilize digital analysis to quantify a difference in methylation-state at a target region of nucleic acid molecules in a mixed sample by a) enzymatically reacting with molecules in the mixed sample having the target region in a methylation state recognized by the enzyme and in the presence of control nucleic acid comprising the target region in the methylation state recognized by the enzyme, b) amplifying the remaining nucleic acid molecules in the sample, and c) quantifying an amount of total amplifiable nucleic acid from the mixed sample, an amount of molecules comprising the target region, and an amount of the control nucleic acid, where the amount of control nucleic acid is used to determine efficiency of the enzymatic reaction and quantify what number of molecules in the amount of molecules comprising the target region are from molecules in a methylation-state that is not recognized by the enzyme.

Methods of the invention account for the fact that, when detecting differences in methylation-state targets using MSRE's, the difference between a failed assay (i.e. inaccurate quantification) and a positive assay (i.e. accurate quantification) is dependent on the degree of enzymatic reaction of target regions of the nucleic acid in a methylation state recognized by the MSRE (e.g. non-methylated recognition site). The present invention overcomes that dependency by normalizing the amount of measured molecules comprising the target region based on a value of the enzymatic reaction efficiency of the MSRE for the target regions in the recognized methylation-state using a multiplexed assay on the same sample.

In certain aspects, methods of the invention involve providing a mixed sample of nucleic acid molecules comprising target regions in different methylation-states, reference nucleic acid molecules that do not comprise the target region, and a first number of control nucleic acid molecules comprising the target region in the recognized methylation-state (e.g. added to the mixed sample in a known concentration), and conducting methylation-specific enzymatic reaction of nucleic acid in a sample. After enzymatic reaction, a number of the molecules comprising the target regions, a number of the reference nucleic acid molecules, and a number of the control nucleic acid molecules is determined. A value of the efficiency of the methylation-specific enzymatic reaction is then measured using the first number of control nucleic acid molecules and second number of unreacted control nucleic acid molecules. The efficiency value is then used to normalize the number of the molecules comprising the target regions to calculate a number of molecules comprising the target region that are derived from molecules in a methylation-state that is not recognized by the enzyme which can then be used with the number of reference nucleic acid molecules to calculate the difference or ratio of molecules comprising the target region in a recognized methylation state versus the number of molecules comprising the target region in an unrecognized state. The ratio can then be used to identify a disease or condition. In addition, the disease or condition may relate to a percent of the mixed sample deriving from a certain source (e.g. a sample taken from an individual that has multiple targets, some of which include state dependent targets). For example, the normalized ratio may be indicative of a percent of fetal DNA in a sample or indicative of the presence/absence of cancer cells from a tissue. In some cases the source of the mixed sample may include DNA obtained from blood that comprises circulating tumor cells (also referred to as CTC) or cell free DNA (also referred to as cfDNA). In some cases the source of the mixed sample may include DNA obtained from saliva, urine, tear, vaginal secretion, amniotic fluid, breast fluid, breast milk, sweat, or other heterogeneous tissue.

A number of examples are described herein that involve performing methylation-specific enzymatic reaction in order to allow for subsequent detection and quantification of differences in methylation state of molecules at one or more target regions in a mixed sample of nucleic acids. However it will be appreciated that other state specific enzymes exist and the same methods described herein may be employed using these enzymes, mixed samples of nucleic acid molecules having state dependent target regions, and control nucleic acid. Therefore the use of MSRE's should not be considered limiting.

It is generally appreciated that MSRE's include, inter alia, MspI and HpaII which cleave nucleic acid molecules at recognition sites based on methylation status (also sometimes referred to as cleavage sites). It will be appreciated that there are a number of different species of MSRE having different recognition sites and have different activity based on different methylation states. For example, some methylation specific enzymes recognize when a specified recognition site is not methylated resulting in cleavage of the nucleic acid where the same methylated recognition site is not recognized and left intact. In embodiments of the described invention it is important that the enzyme is specific for either the completely modified state or completely unmodified state and not a state where partial modification is recognized by the enzyme (e.g. completely methylated or completely un-methylated in the example of MSRE's). However, it is generally appreciated that the enzymatic activity can be inefficient for a variety of reasons resulting in an incomplete reaction of the recognized nucleic acid molecules in a sample and thus exact quantification of the methylation-state differences of targets can be inaccurate and unreliable without compensating for enzyme efficiency.

As described above, in order to provide accurate quantification of the methylation-state differences within a mixed sample, methods of the invention provide for assessing the efficiency of the methylation-specific enzymatic reaction. The efficiency of the methylation-specific enzymatic reaction in an assay, according to certain embodiments, can be measured by adding a known amount of control nucleic acid molecules to the sample prior to performing methylation specific enzymatic reaction. In the described embodiments, the control nucleic acid molecule includes at least one recognition site in a methylation state that is recognized by the MSRE employed in the assay. After enzymatic reaction the remaining molecules may be amplified (e.g. via digital PCR) and the efficiency of the enzymatic reaction by quantified by counting the number of control nucleic acid molecules present in the sample that should have been enzymatically reacted, and comparing that number to the known amount of control nucleic acid molecules introduced to prior to enzymatic reaction. For example, the MSRE cleavage site of the control nucleic acid is in a recognized methylation state, where an absence of control nucleic acid molecules after enzymatic reaction indicates that enzymatic reaction is complete, and a presence of control nucleic acid after enzymatic reaction indicates that enzymatic reaction is incomplete. For incomplete enzymatic reaction, a number of control nucleic acid remaining after enzymatic reaction relative to the known amount of control nucleic acid added to the assay is used to measure the efficiency of the enzymatic reaction (e.g. percent efficiency). The measured efficiency can then be used to normalize a value of methylation-dependent targets in the assay.

In certain embodiments, one or more MSRE's that recognize one or more regions that differ in methylation state by disease or condition of an individual (or individuals in the case of prenatal testing for aneuploidy) as well as recognizing one or more regions in control nucleic acid molecules comprising the methylation state recognized by the MSRE can be introduced into the mixed sample including the target nucleic acid from the individual(s) for detection of the disease or condition, a reference nucleic acid from the individual, and control nucleic acid not associated with the individual added to the mixed sample in a known quantity. The MSRE's are used to enzymatically react with one or more regions of the target nucleic acid molecules in the recognized methylation state, thereby leaving one or more regions of target nucleic acid molecules that are not in the recognized methylation state intact for subsequent amplification and/or detection. In the same sample, the MSRE's are used to enzymatically react with the regions of the control nucleic acid molecules in the recognized methylation state, as discussed above, in order to determine the efficiency of the enzymatic reaction. In embodiments of the invention described herein, the reference molecules are never a substrate for enzymatic reaction and the control nucleic acid molecules are always a substrate for enzymatic reaction. The number of intact target nucleic acid molecules are subsequently quantified using the number of intact control nucleic acid molecules to normalize for the enzymatic efficiency.

In certain aspects, methods of the invention rely on counting techniques to quantify a ratio or fraction of intact target nucleic acid molecules relative to the number reference nucleic acid molecules. The ratio is indicative of a disease or condition. According to some embodiments, the method provides for counting nucleic acids in partitioned compartments, such as droplets, as in digital PCR. This method involves partitioning the nucleic acid from a sample, after the enzymatic reaction step, into droplets such that a subset of droplets includes nucleic acid from the mixed sample, and amplifying nucleic acid within each droplet. In some embodiments the modification state specific enzymes are included in the mixed sample partitioned into the droplets so that enzymatic reaction step occurs in the droplets, along with any subsequent reactions for processing and detection. The amplified product in each droplet may be detected by, for example, a hydrolysis probe-based assay. In some embodiments, the droplets contain one or fewer target nucleic acid molecules per droplet. In the same or alternative embodiments the nucleic acid molecules partitioned into droplets may include one or more multiplexed target molecules comprising one or more regions recognized by one or more state specific enzymes that differs in state by disease or condition of an individual, one or more reference nucleic acid molecules, and one or more control nucleic acid molecules. In addition, components for an amplification reaction and detection (e.g. probes with detectable labels, primers, reagents) may be introduced into the droplets. The probes with detectable labels and primers introduced into each droplet may be specific for different targets.

An exemplary method for forming droplets involves flowing a stream of aqueous fluid including the mixed sample such that it intersects two opposing streams of flowing carrier fluid that is immiscible with the aqueous fluid. Intersection of the aqueous fluid with the two opposing streams of flowing carrier fluid results in partitioning of the aqueous fluid into individual aqueous droplets. The carrier fluid may be any fluid that is immiscible with the aqueous fluid. An exemplary carrier fluid is oil, particularly, a fluorinated oil. In certain embodiments, the carrier fluid includes a surfactant, such as a fluorosurfactant. The droplets may be flowed through channels.

Detection of the nucleic acid molecules that include molecules with target regions that differ in methylation state, reference molecules, and control nucleic acid molecules can be accomplished by introducing a plurality of probe species that identify the particular nucleic acid types to the mixed sample. Typically the probe species are added to the mixed sample after the enzymatic reaction steps are complete and subsequently included in the partitioned compartments, although as described above in some embodiments the probe species may be included in the mixed sample with the state specific enzyme when partitioned. Members of the plurality of probe species can each include the same detectable label, or a different detectable label. The detectable label is preferably a fluorescent label. The plurality of probe species can include one or more groups of probe species at varying concentrations. The one or more groups of probe species can include the same detectable label which varies in intensity upon detection, due to the varying probe concentrations. In some embodiments, detecting the label may be accomplished by an amplification based technique, such as PCR, digital PCR, or qPCR. In specific embodiments, digital PCR is used to detect the labels. Similarly for amplification, one or more sets of primer pairs may be introduced into the assay after enzymatic reaction. Each set of primer pairs capable of amplifying a specific region of interest from the nucleic acid molecules from an individual (e.g. region comprising the state specific targets and/or reference region) and control nucleic acid molecule.

In certain aspects, embodiments of the invention involve screening for a disease or condition. Screening for a disease or condition may involve counting a first number of compartmentalized portions including a first detectable label that identifies one or more state specific targets, counting a second number of compartmentalized portions comprising a second detectable label corresponding to reference targets, adjusting the first number corresponding to the state specific targets based on the efficiency of a enzyme activity; and determining whether a statistical difference exists between adjusted first number and the second number.

### BRIEF DESCRIPTION OF THE DRAWINGS

FIG. 1 depicts a droplet formation device.

FIG. 2 depicts a portion of the droplet formation device of FIG. 1.

FIGS. 3A-3C depict an exemplary microfluidic system for droplet generation and readout. FIG. 3A depicts the droplet generation chip; FIG. 3B depicts the droplet spacing for readout; and FIG. 3C depicts a cartoon of droplet readout by fluorescence.

FIGS. 4A-4C depict the serial dilution of template DNA quantified by dPCR. FIG. 4A shows droplet fluorescence during readout for the most concentrated sample. Each discrete burst of fluorescence corresponded to an individual droplet. Two different groups of droplets were evident: PCR(+) droplets peaking at ˜0.8 V and PCR(−) droplets at ˜0.1 V; FIG. 4B shows a histogram of the peak fluorescence intensities of droplets from the complete data trace in (a). PCR(+) and PCR(−) droplets appeared as two very distinct populations centered at 0.78 and 0.10 V, respectively; FIG. 4C shows the serial dilution of template DNA. Open circles: measured occupancies; solid line: the best fit to Eqn 2 (A=0.15, ƒ=4.8, R²—0.9999).

FIGS. 5A-5E are a series of schematic depicting one-color detection of a genetic sequence with a microfluidic device.

FIG. 6 illustrates a digital PCR 3-plex assay according to certain embodiments.

FIG. 7 illustrates a digital PCR 4-plex assay according to certain embodiments.

### DETAILED DESCRIPTION

The invention generally relates to methods for analyzing nucleic acid in a sample. In certain aspects, methods of the invention involve providing a mixed sample of nucleic acid comprising modification state specific targets, reference targets, and a first number of control nucleic acid molecules, and conducting modification state specific enzymatic reaction of nucleic acid in a sample using target/substrate state dependent enzymes. After enzymatic reaction, a number of the intact modification state specific targets, a number of the reference targets, and a second number of the control nucleic acid molecules is determined. A value of the completeness/efficiency of the modification state specific enzymatic reaction is then determined based on the first and second numbers of the control nucleic acid molecules. The efficiency value is then used to normalize the number of intact modification state specific targets counted and/or a ratio of the number of the modification state specific target molecules counted and the number of reference molecules counted. The ratio can then be used to identify a disease or condition. In some embodiments, the disease or condition may relate to embryogenesis, epigenetic regulation, or a disease. In the same or alternative embodiments, the disease or condition may relate to a percent of sample deriving from a certain source. For example, the normalized ratio may be indicative of a percent of fetal DNA in a sample relative to the maternal DNA, or percent of tumor DNA relative to normal DNA. Importantly, the normalized ratio may include a percentage of DNA in a first modification state relative to DNA in a second modification state that occurs in a heterogeneous sample.

The term “modification” of nucleic acid molecules, as used herein, includes any type of modification useable as a marker between a modified and unmodified state, for example, methylation and histone acetylation (e.g. chromatin). In the embodiments described herein, the nucleic acid modification is subject to enzymatic reaction by one or more target/substrate state specific enzymes and thus the modification state of the nucleic acid can be employed to selectively enrich for a population in one state versus a second population in another state. In some embodiments, some enzymes may add a modification a substrate in a state specific manner rather than remove the substrate from the sample, where the modification may be detectable or play a role in the process of detection. For example, embodiments of the invention include any target substrate that can be differentiated by an enzyme which can produce a product in a state specific manner. Other examples could include proteases, lipases, ligases, polymerases, restriction enzymes, 3-OH MSRE, and chromatin motif specific enzymes.

In the described embodiments, nucleic acid molecules in a sample may include one or more modification state specific target regions and one or more reference target regions where enzymatic treatment is employed to “enrich” for molecules comprising the state specific target region in a state not recognized and enzymatically reacted by the state specific enzyme that enables accurate quantification of differences of modification state or imbalances. An important element in the described embodiments includes a ‘spike-in’ exogenous control nucleic acid that is added in a specific quantity such that following enzymatic treatment of the sample the extent of enzymatic reaction is precisely quantified by counting the remaining number of spiked-in control molecules. It is again important to note that the control nucleic acid molecule is always a substrate for enzyme reaction in the described embodiments. For example, the control nucleic acid comprises one or more regions in the correct modification state that is recognized by the state specific enzyme and enzymatically reacted. A value for the extent of enzymatic reaction is used to determine a ‘normalizing factor’ for quantification of the number of molecules that should not be subject to enzymatic reaction.

For example, a methylation state specific target region comprises one or more substrate/recognition sites for at least one MSRE and resides, all or in part, within a target region or locus of a nucleic acid where the presence or absence of methylation at the locus is population dependent and where a difference between populations indicates the presence of a disease or condition. Further, the methylation state specific target region comprises one or more sets of sites that are recognized by primers and probes where the sets of sites may be the same as or different from the recognition site(s) for the MSRE. A methylation state independent reference molecule comprises one or more endogenous control regions that is independent of the enzymatic treatment for quantification of the amount of input molecules from an individual in the mixed sample (e.g. counting a target that is not a substrate/recognition site for the enzyme(s)). In the presently described example, if the target/assay is “methylation state independent”, containing no potential MSRE cleavage sites, then counting the number of this target with a digital amplification technique (such as digital PCR) assay results in a methylation state independent cluster on a 2-D scatter plot that indicates the number of “genome equivalents” of DNA in the sample. The number of genome equivalents is useful in the calculation of the difference between the number of modified versus unmodified molecules in a sample comprising a mixture of both species (e.g. a mixed or heterogeneous sample). For instance, in the case where an MSRE recognizes the un-methylated state of a recognition site, the number of reference molecules representing the genome equivalents will be the denominator and the number of target molecules counted in the methylated state the numerator in the fraction for counting the number of methylated target loci molecules per genomic equivalent in the sample.

Embodiments of a reference molecule comprises a region of a nucleic acid molecule that do not have a recognition site for any the enzymes used to enzymatically react with the methylation dependent targets. It is again important to note that the reference molecule is never a substrate for enzyme reaction in the described embodiments. In addition, a reference molecule is selected due its lack of copy number variation so that the number of genome equivalents in the mixed sample can be easily and accurately assessed. In certain embodiments, multiple different methylation state dependent targets, reference molecules, and control nucleic acid molecules can be assessed in a single assay. The reference nucleic acid can be used to determine a total amount of amplifiable genome equivalents in a sample as compared to an amount of the methylation state specific targets.

Methods of the invention involve performing modification state specific enzymatic reaction in order to allow for subsequent detection of modification state specific targets in nucleic acid. One example includes methylation state specific enzymatic reaction that utilizes MSRE's that cleave recognition sites having specific sequence composition based on methylation status. For example, some species of MSRE recognize an un-methylated site and cleaves the nucleic acid resulting in enzymatic reaction with the un-methylated target nucleic acid and leave molecules comprising the methylated recognition sites of methylation state specific targets intact. The intact molecules can then be used in subsequent processing steps such as amplification and quantification. However in the present example, the MSRE enzymatic reaction may not be 100% efficient resulting in inefficient and incomplete enzymatic reaction of the un-methylated target regions, and thus the number of intact molecules counted from the methylation state specific target molecules includes the expected methylated population and some number of unexpected un-methylated molecules counted as false positives. Therefore, in order to accurately quantify the number of modification state specific targets in the particular state, embodiments of the invention provide for assessing the efficiency of the modification-specific enzymatic reaction.

The efficiency of the modification-specific enzymatic reaction in an assay, according to certain embodiments, can be measured by adding a known amount of control nucleic acid to the assay prior to performing modification-specific enzymatic reaction. In the described embodiments, the control nucleic includes a modification-specific restriction enzyme cleavage site and is in a modification state that is recognized by the enzyme for enzymatic reaction. In the described embodiments, the number of the control nucleic acid molecules remaining after the enzymatic reaction step are counted and compared to the known amount of control nucleic acid added prior to enzymatic reaction to determine a measure of the efficiency of enzymatic reaction. For example, in some embodiments the modification-specific restriction enzyme cleavage site of the control nucleic acid is non-modified and recognized for cleavage by the enzyme so that an absence of control nucleic acid after enzymatic reaction indicates that the enzymatic reaction is complete, whereas a presence of some amount of control nucleic acid after enzymatic reaction indicates that the enzymatic reaction is incomplete. For incomplete enzymatic reaction, a number of control nucleic acid remaining after the enzymatic reaction relative to the known amount of control nucleic acid added to the assay is used to measure the efficiency of the enzymatic reaction. The measured efficiency can then be used to normalize a value of modification state specific targets of unknown starting quantity within the assay. That is, the measured efficiency can be used to normalize an amount of methylation state specific targets in a sample. For instance, the number of modification state specific targets counted after enzymatic reaction may be adjusted based on the measured efficiency of the enzymatic reaction corresponding to the number of unreacted control nucleic acid molecules counted. A ratio of the adjusted number of modification-dependent targets to the number of reference targets is then determined. Alternatively, ratio of the number of modification-dependent targets to the number of reference targets is determined first, and then the ratio is adjusted based the measured efficiency of the enzymatic reaction corresponding to the amount of the control nucleic acid. The ratio may be indicative of a disease or condition.

The normalization process using the measure of enzymatic reaction efficiency is very important for the precision of the assay and confidence that the number of molecules counted as false positive or false negative are minimized. The precision of the assay is particularly important when the number of molecules in a certain modification state in the sample available for counting is very low. For example, a heterogeneous sample may comprise a fraction of target nucleic acid molecules in a certain modification state associated with a disease or condition, where the fraction is less than 5%, less than 2% or less than 1% of the heterogeneous sample. In the present example the heterogeneous sample may be employed in prenatal testing for genetic aneuploidy where the fetal fraction of DNA can range from about 2-10%, or less than 2% of the DNA in the heterogeneous sample. Typically, an expected ratio of maternal to fetal chromosomes would be expected to be 1:1 for a normal fetus. However, in the case where a fetal aneuploid condition exists, the statistics for accurately identifying the aneuploid condition depend heavily on the amount of the fetal fraction of DNA with the chromosomal abnormality (e.g. particularly if low, ˜3% or less) and/or when the total number of countable molecules in the heterogeneous sample is low. In such cases precision of the assay is statistically important in order to accurately call the existence of the aneuploid condition. In other words, a low fetal fraction from a sample with a low number of countable molecules can result in a small difference in calculated ratio where the precision of detection provides measure of statistical confidence that the small difference in ratio can be accurately called as an aneuploid condition.

It will be appreciated that prenatal testing is provided in the example above, however the same applies to cancer analysis, or other assay of heterogeneous tissues using embodiments of the described invention. Therefore, the example of prenatal testing should not be considered as limiting.

Some embodiments of the invention involve determining a number of target nucleic acid molecules in the sample, such as by determining a number of modification state specific targets, determining a number of reference molecules, and determining a number of control nucleic acid molecules. For example, the numbers of molecules may be determined by detecting target-specific detectable labels that may be coupled to probes, such as hydrolysis probes.

In one aspect, the invention provides droplets containing a single nucleic acid template or fewer, amplification reagents, and one or more detectable probes. The amplification reagents may include multiplexed PCR primers that along with the detectable probes may be specific to the modification-dependent target(s), reference target(s), or control nucleic acid(s). Methods of the invention also provide for detecting the nucleic acid template by forming such droplets and amplifying the nucleic acid templates using droplet based digital amplification.

Reactions within microfluidic droplets may yield very uniform fluorescence intensity at the end point, and ultimately the intensity depends on the efficiency of probe hydrolysis. Thus, in another aspect of the methods of the invention, different reactions with different efficiencies can be discriminated on the basis of end point fluorescence intensity alone even if they have the same color. Furthermore, in another method of the invention, the efficiencies can be tuned simply by adjusting the probe concentration, resulting in an easy-to-use and general purpose method for multiplexing. In another aspect of the invention, adding multiple colors increases the number of possible reactions geometrically, rather than linearly as with qPCR, because individual reactions can be labeled with multiple fluorophores.

### Nucleic Acids

Nucleic acid is generally is acquired from a sample or a subject. Target molecules for labeling and/or detection according to the methods of the invention include, but are not limited to, genetic and proteomic material, such as DNA, genomic DNA, RNA, expressed RNA, chromosomal and/or extra-chromosomal targets (e.g. mitochondrial, episomal, exosomal). Methods of the invention are applicable to DNA from whole cells or to portions of genetic or proteomic material obtained from one or more cells, or cell-free DNA. For a subject, the sample may be obtained in any clinically acceptable manner, and the nucleic acid templates are extracted from the sample by methods known in the art. Nucleic acid templates can be obtained as described in U.S. Patent Application Publication Number US2002/0190663 A1, published Oct. 9, 2003. Generally, nucleic acid can be extracted from a biological sample by a variety of techniques such as those described by Maniatis, et al. (Molecular Cloning: A Laboratory Manual, Cold Spring Harbor, N.Y., pp. 280-281, 1982), the contents of which are incorporated by reference herein in their entirety.

Nucleic acid templates include deoxyribonucleic acid (DNA) and/or ribonucleic acid (RNA). Nucleic acid templates can be synthetic or derived from naturally occurring sources. In one embodiment, nucleic acid templates are isolated from a biological sample containing a variety of other components, such as proteins, lipids and non-template nucleic acids. Nucleic acid templates can be obtained from any cellular material, obtained from an animal, plant, bacterium, fungus, or any other cellular organism. Biological samples for use in the present invention include viral particles or preparations. Nucleic acid templates can be obtained directly from an organism or from a biological sample obtained from an organism, e.g., from blood, urine, cerebrospinal fluid, seminal fluid, saliva, sputum, stool and tissue. In a particular embodiment, nucleic acid is obtained from fresh frozen plasma (FFP). Any tissue or body fluid specimen may be used as a source for nucleic acid for use in the invention. Nucleic acid templates can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

In some embodiments nucleic acid molecules obtained from biological samples is fragmented to produce suitable template molecules for analysis. In one embodiment, nucleic acid from a biological sample is fragmented by sonication. It will however be appreciated that there are numerous methods for fragmenting nucleic acid molecules known in the related art and any method may be used with the presently described embodiments.

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C6H4-(OCH2-CH2)xOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), .beta.-mercaptoethanol, DTE, GSH, cysteine, cysteamine, tricarboxyethyl phosphine (TCEP), or salts of sulfurous acid. Once obtained, the nucleic acid is denatured by any method known in the art to produce single stranded nucleic acid templates and a pair of first and second oligonucleotides is hybridized to the single stranded nucleic acid template such that the first and second oligonucleotides flank a target region on the template.

Nucleic acid molecules within a sample may include modified regions as discussed above. For example, prokaryotic nucleic acid is methylated at cytosine and adenosine residues (see, e.g., McClelland et al., Nuc. Acids. Res. 22:3640-3659 (1994). Methylation of prokaryotic nucleic may protect the nucleic from enzymatic reaction by cognate restriction enzymes, i.e., foreign DNAs (which are not methylated in this manner) that are introduced into the cell are enzymatically reacted by restriction enzymes which cannot enzymatically react with the methylated prokaryotic nucleic acid. Nucleic acid methylation patterns can be used to identify specific bacterial types (e.g., genus, species, strains, and isolates).

Mammalian nucleic acid is often methylated at cytosine residues, typically these cytosines are 5′ neighbors of guanine (CpG). This methylation has been shown by several lines of evidence to play a role in gene activity, cell differentiation, tumorigenesis, X-chromosome inactivation, genomic imprinting and other major biological processes (Razin and Riggs eds. in DNA Methylation Biochemistry and Biological Significance, Springer-Verlag, N.Y., 1984). Research suggests that genes with high levels of methylation in a promoter region are transcriptionally silent, which may allow unchecked cell proliferation. When a promoter region has excessive methylation, the methylation is typically most prevalent in sequences having CpG repeats, so called “CpG islands.” Undermethylation (hypomethylation) has also been implicated in the development and progression of cancer through different mechanisms.

In eukaryotic cells, methylation of cytosine residues that are immediately 5′ to a guanosine, occurs predominantly in CG poor loci (Bird, Nature 321:209 (1986)). In contrast, discrete regions of CG dinucleotides called CpG islands remain unmethylated in normal cells, except during X-chromosome inactivation and parental specific imprinting (Li, et al., Nature 366:362 (1993)) where methylation of 5′ regulatory regions can lead to transcriptional repression.

Aberrant methylation, including aberrant methylation at specific loci, is often associated with a disease state. For example, de novo methylation of the Rb gene has been demonstrated in a small fraction of retinoblastomas (Sakai, et al., Am. J. Hum. Genet., 48:880 (1991)), and a more detailed analysis of the VHL gene showed aberrant methylation in a subset of sporadic renal cell carcinomas (Herman, et al., PNAS USA, 91:9700 (1994)). Expression of a tumor suppressor gene can also be abolished by de novo DNA methylation of a normally unmethylated 5′ CpG island. See, e.g., Issa, et al., Nature Genet. 7:536 (1994); Merlo, et al., Nature Med. 1:686 (1995); Herman, et al., Cancer Res., 56:722 (1996); Graff, et al., Cancer Res., 55:5195 (1995); Herman, et al., Cancer Res. 55:4525 (1995). Methylation of the p16 locus is associated with pancreatic cancer. See, e.g., Schutte et al., Cancer Res. 57:3126-3131 (1997). Methylation changes at the insulin-like growth factor II/H19 locus in kidney are associated with Wilms tumorigenesis. See, e.g., Okamoto et al., PNAS USA 94:5367-5371 (1997). The association of alteration of methylation in the p15, E-cadherin and von Hippel-Lindau loci are also associated with cancers. See, e.g., Herman et al., PNAS USA 93:9821-9826 (1997). The methylation state of GSTP 1 is associated with prostate cancer. See, e.g., U.S. Pat. No. 5,552,277. Tumors where certain genomic loci are methylated have been found to respond differently to therapies such as cis-platin or radiation treatment than tumors where the same genomic loci are un-methylated. It is clear that DNA from tumor cells at certain genomic loci can be different in the levels of DNA methylation and in this way can be distinguished from the DNA from adjacent normal cells. DNA from tumor cells has been found in various body fluids and other clinical specimens collected from cancer patients. For example, methylated DNA having the same sequence of tumor suppressor genes has been found in serum, urine, saliva, sputum, semen, lavages, cell scrapes, biopsies, resected tissues, and feces. Therefore, detection of altered methylation profiles at loci where such alterations are associated with disease can be used to provide diagnoses or prognoses of disease.

Other examples include aberrantly methylated SEPT9 DNA in plasma correlated with occurrence of colorectal cancer (See deVos et al. Clin Chem. 2009 July;55 (7):1337-46); MGMT promoter methylation predictive of response to radiotherapy and chemotherapy (See Rivera et al Neuro Oncol (2010) 12 (2): 116-121); and RASSF1A implicated in cancer (See Dis Markers. 2007;23 (1-2):73-87) as well as a fetal marker.

It will also be appreciated the RNA may exhibit differences in methylation state and is considered within the scope of the described embodiments.

### Modification-Specific Enzymatic Reaction

Embodiments of the invention provides for performing modification specific enzymatic reaction of nucleic acid in the sample. In some cases, enzymatic reaction is performed prior to partitioning the nucleic acid for digital analysis, however enzymatic reaction may also be performed within the partitions. In some embodiments methylation-specific enzymatic reaction is used to enrich for a particular target-state to enable accurate quantification. For example, enrichment of one target-state (e.g. methylated or un-methylated) improves the precision and confidence of statistical accuracy of numbers derived from counting target copy numbers in a heterogeneous mixture (e.g. if a completely enriched 100% fetal fraction is prepared, then one needs to see a 50% increase in counts of chromosome 21 targets compared to a normal diploid chromosome target, whereas if the fetal fraction is 10% then one needs to see a 5% increase to call out an aneuploidy condition such as trisomy). The amount of input material and the precision of the measurement are very much improved by using enriched material.

The enrichment occurs from methylation-specific enzymatic reaction that prevents a target locus in a methylation state from being amplified such that target loci in the other methylation state are amplified and detected. In addition, methylation-specific enzymatic reaction is also used to enzymatically react with a known quantity of control nucleic acid, placed in the sample, containing MSRE cleavage sites in the recognized methylation state. As discussed, a comparison of the quantity of the control nucleic acid molecules after enzymatic reaction and the known quantity of control nucleic acid molecules placed in the sample allows one to determine the efficiency of the enzymatic reaction.

Methylation-specific enzymatic reaction techniques are known in the art, and utilize MSRE's. Some species of methylation-specific restriction enzymes cleave DNA at or in proximity to an un-methylated recognition sequence but does not cleave at or in proximity to the same sequence when the recognition sequence is methylated. Alternatively, some species of MSRE's cleave DNA at or in proximity to a methylated recognition sequence but does not cleave at or in proximity to the same sequence when the recognition sequence is un-methylated. Exemplary MSRE's are described in, e.g., McClelland et al., Nucleic Acids Res. 22 (17):3640-59 (1994) and http://rebase.neb.com. In addition, MSRE's are described in PCR: Methods Express: Chapter 17 PCR-based methods to determine DNA methylation status at specific CpG sites using methylation-specific restriction enzymes. (S. Hughes and A. Moody, eds., 2007). Another PCR-based process that involves enzymatic reaction of genomic DNA with MSRE's prior to PCR amplification is described in Singer-Sam et al., Nucl. Acids Res. 18:687,1990.

Suitable methylation-specific restriction enzymes that do not cleave DNA at or near their recognition sequence when a cytosine within the recognition sequence is methylated at position C5 include, e.g., Aat II, Aci I, Acl I, Age I, Alu I, Asc I, Ase I, AsiS I, Bbe I, BsaA I, BsaH I, BsiE I, BsiW I, BsrF I, BssH II, BssK I, BstB I. BstN I, BstU I, Cla I, Eae I, Eag I, Fau I, Fse I, Hha I, HinP1 I, HinC II, Hpa II, Hpy99 I, HpyCH4 IV, Kas I, Mbo I, Mlu I, MapA1 I, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, and Zra L Suitable methylation-specific restriction enzymes that do not cleave DNA at or near their recognition sequence when an adenosine within the recognition sequence is methylated at position N6 include, e.g., Mho I.

As discussed, control nucleic acids are nucleic acids that include methylation-specific cleavage sites in the recognized methylation state (e.g. always a substrate for enzymatic reaction). In the embodiments described herein, the control nucleic acids may include synthesized molecules, or purified control molecules. As one skilled in the art would appreciate, any nucleic acid molecule with a MSRE cleavage site can be used with its associated MSRE. In some embodiments, the cleavage site of the control nucleic acid is not methylated, such that the appropriate MSRE enzymatically reacts with all of the known control nucleic acid in the sample. This indicates that the enzymatic reaction is complete. If any of the control nucleic acid in the nucleic acid is not enzymatically reacted, unreacted control nucleic acid molecules are detected and quantified. The number of unreacted control nucleic acid molecules are then compared known amount of control nucleic acid molecules introduced to the sample prior to enzymatic reaction. The ratio of unreacted control molecules to the known amount of control molecules can be used as a measure of the efficiency or completeness of the methylation-specific enzymatic reaction.

### Droplet Formation

Methods of the invention involve forming sample droplets where some droplets contain zero template nucleic acid molecules, some droplets contain one template nucleic acid molecule, and some droplets may or may not contain multiple template nucleic acid molecules. In some embodiments, a sample is divided into compartments such that only one or fewer of either reference target, methylation-dependent target, or control target is in any one droplet. In the described embodiments, the distribution of target nucleic acid molecules within droplets obeys the Poisson distribution. However, methods for non-Poisson loading of droplets are known to those familiar with the art, and include but are not limited to active sorting of droplets, such as by laser-induced fluorescence, or by passive one-to-one loading. The description that follows assumes Poisson loading of droplets, but such description is not intended to exclude non-Poisson loading, as the invention is compatible with all distributions of DNA loading.

The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety.

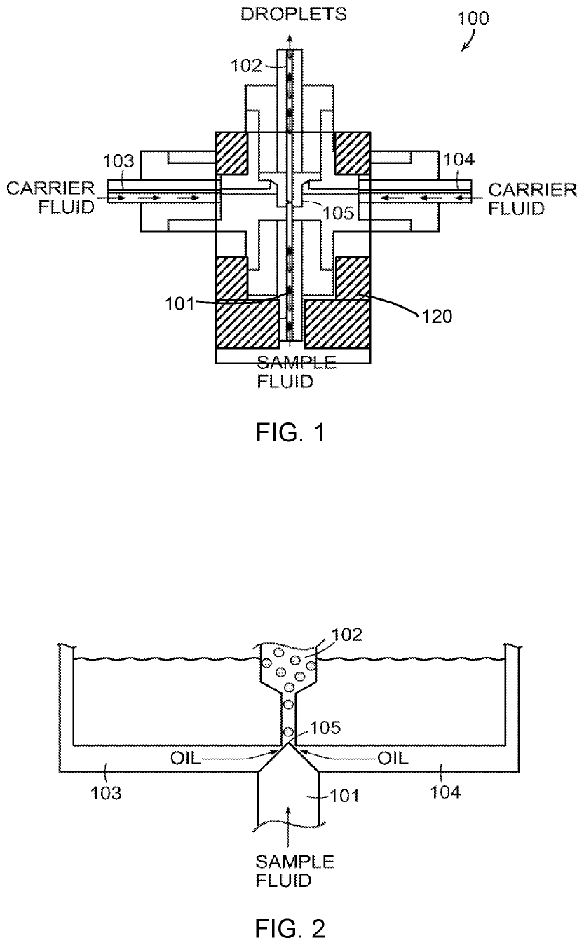

FIG. 1 shows an exemplary embodiment of a device 100 for droplet formation. Device 100 includes an inlet channel 101, an outlet channel 102, and two carrier fluid channels 103 and 104. Device 100 may additionally comprise a temperature block 120 to control the temperature of the fluids during droplet formation. Temperature block 120 may be used to heat or cool the fluids as needed, and may be connected to a temperature controller (not shown) to control the temperature during droplet formation. Channels 101, 102, 103, and 104 meet at a junction 105. Inlet channel 101 flows sample fluid to the junction 105. Carrier fluid channels 103 and 104 flow a carrier fluid that is immiscible with the sample fluid to the junction 105. Inlet channel 101 narrows at its distal portion wherein it connects to junction 105 (see FIG. 2). Inlet channel 101 is oriented to be perpendicular to carrier fluid channels 103 and 104. Droplets are formed as sample fluid flows from inlet channel 101 to junction 105, where the sample fluid interacts with flowing carrier fluid provided to the junction 105 by carrier fluid channels 103 and 104. Outlet channel 102 receives the droplets of sample fluid surrounded by carrier fluid.

The sample fluid is typically an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid is one that is immiscible with the sample fluid. The carrier fluid can be a non-polar solvent, decane (e g., tetradecane or hexadecane), fluorocarbon oil, silicone oil or another oil (for example, mineral oil).

In certain embodiments, the carrier fluid contains one or more additives, such as agents which increase, reduce, or otherwise create non-Newtonian surface tensions (surfactants) and/or stabilize droplets against spontaneous coalescence on contact. Surfactants can include Tween, Span, fluorosurfactants, and other agents that are soluble in oil relative to water. In some applications, performance is improved by adding a second surfactant, or other agent, such as a polymer or other additive, to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing.

In certain embodiments, the droplets may be coated with a surfactant or a mixture of surfactants. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglycerl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

In certain embodiments, the carrier fluid may be caused to flow through the outlet channel so that the surfactant in the carrier fluid coats the channel walls. In one embodiment, the fluorosurfactant can be prepared by reacting the perflourinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., FLUORINERT (3M)), which then serves as the carrier fluid.

One approach to merging sample fluids, using a device called a lambda injector, involves forming a droplet, and contacting the droplet with a fluid stream, in which a portion of the fluid stream integrates with the droplet to form a mixed droplet. In this approach, only one phase needs to reach a merge area in a form of a droplet. Further description of such method is shown in pending U.S. patent application Ser. No. 13/371,222, the content of which is incorporated by reference herein in its entirety.

According to a method for operating the lambda injector, a droplet is formed as described above. After formation of the sample droplet from the first sample fluid, the droplet is contacted with a flow of a second sample fluid stream. Contact between the droplet and the fluid stream results in a portion of the fluid stream integrating with the droplet to form a mixed droplet.

The droplets of the first sample fluid flow through a first channel separated from each other by immiscible carrier fluid and suspended in the immiscible carrier fluid. The droplets are delivered to the merge area, i.e., junction of the first channel with the second channel, by a pressure-driven flow generated by a positive displacement pump. While droplet arrives at the merge area, a bolus of a second sample fluid is protruding from an opening of the second channel into the first channel. Preferably, the channels are oriented perpendicular to each other. However, any angle that results in an intersection of the channels may be used.

The bolus of the second sample fluid stream continues to increase in size due to pumping action of a positive displacement pump connected to channel, which outputs a steady stream of the second sample fluid into the merge area. The flowing droplet containing the first sample fluid eventually contacts the bolus of the second sample fluid that is protruding into the first channel. Contact between the two sample fluids results in a portion of the second sample fluid being segmented from the second sample fluid stream and joining with the first sample fluid droplet to form a mixed droplet. In certain embodiments, each incoming droplet of first sample fluid is merged with the same amount of second sample fluid.

In certain embodiments, an electric charge is applied to the first and second sample fluids. Description of applying electric charge to sample fluids is provided in Link et al. (U.S. patent application number 2007/0003442) and European Patent Number EP2004316 to Raindance Technologies Inc, the content of each of which is incorporated by reference herein in its entirety. Electric charge may be created in the first and second sample fluids within the carrier fluid using any suitable technique, for example, by placing the first and second sample fluids within an electric field (which may be AC, DC, etc.), and/or causing a reaction to occur that causes the first and second sample fluids to have an electric charge, for example, a chemical reaction, an ionic reaction, a photocatalyzed reaction, etc.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. The electric field generator may be constructed and arranged to create an electric field within a fluid contained within a channel or a microfluidic channel. The electric field generator may be integral to or separate from the fluidic system containing the channel or microfluidic channel, according to some embodiments.

Techniques for producing a suitable electric field (which may be AC, DC, etc.) are known to those of ordinary skill in the art. For example, in one embodiment, an electric field is produced by applying voltage across a pair of electrodes, which may be positioned on or embedded within the fluidic system (for example, within a substrate defining the channel or microfluidic channel), and/or positioned proximate the fluid such that at least a portion of the electric field interacts with the fluid. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof. In some cases, transparent or substantially transparent electrodes can be used.

The electric field facilitates rupture of the interface separating the second sample fluid and the droplet. Rupturing the interface facilitates merging of bolus of the second sample fluid and the first sample fluid droplet. The forming mixed droplet continues to increase in size until it a portion of the second sample fluid breaks free or segments from the second sample fluid stream prior to arrival and merging of the next droplet containing the first sample fluid. The segmenting of the portion of the second sample fluid from the second sample fluid stream occurs as soon as the shear force exerted on the forming mixed droplet by the immiscible carrier fluid overcomes the surface tension whose action is to keep the segmenting portion of the second sample fluid connected with the second sample fluid stream. The now fully formed mixed droplet continues to flow through the first channel.

In other embodiments, the rupture of the interface can be spontaneous, or the rupture can be facilitated by surface chemistry. The invention is not limited in regard to the method of rupture at the interface, as rupture can be brought about by any means.

In the context of PCR, in a preferred embodiment, the first sample fluid contains template molecules of methylation state specific targets, reference targets, and control nucleic acid. The first sample fluid contains the template molecules of methylation state specific target nucleic acid, reference target nucleic acid, and control nucleic acid after methylation state specific enzymatic reaction. The methylation state specific enzymatic reaction is designed to cleave corresponding un-methylated sites of the methylation state specific target nucleic acid, and the methylation state specific enzymatic reaction of the control nucleic acid acts a measure of enzymatic reaction efficiency as discussed previously. The control nucleic acid can be added manually to the first sample fluid or introduced automatically by, for example, merging another fluid stream with the control nucleic acid templates into the first fluid stream. Droplets of the first sample fluid are formed as described above. Those droplets will include template molecules of methylation state specific targets, reference targets, and control nucleic acid molecules. In certain embodiments, some of the droplets will include only a single methylation state specific target molecule, reference target molecule, or control nucleic acid template while other droplets contain no template molecule, and thus digital PCR can be conducted. In a preferred embodiment, the droplets are formed in the presence of reagents and enzymes needed for subsequent PCR reactions. In other embodiments, a second sample fluid contains reagents for the PCR reaction. Such reagents generally include Taq polymerase, deoxynucleotides of type A, C, G and T, magnesium chloride, and forward and (optionally) reverse primers, all suspended within an aqueous buffer. The second fluid also includes detectably labeled probes for detection of the amplified methylation-dependent target, reference target, and control nucleic acid molecule. In an embodiment in which the PCR reagents are in a separate droplet, a droplet containing the nucleic acid is caused to merge with the PCR reagents in the second fluid as described above, producing a droplet that includes Taq polymerase, deoxynucleotides of type A, C, G and T, magnesium chloride, forward and reverse primers, detectably labeled probes, and the target nucleic acid. In another embodiment, the first fluid can contain the template DNA and PCR master mix (defined below), and the second fluid can contain the forward and reverse primers and the probe. The invention is not restricted in any way regarding the constituency of the first and second fluidics for PCR or digital PCR. For example, in some embodiments, the template DNA is contained in the second fluid inside droplets.

### Target Amplification

Methods of the invention further involve amplifying the target nucleic acid in each droplet. Amplification refers to production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). The amplification reaction may be any amplification reaction known in the art that amplifies nucleic acid molecules, such as polymerase chain reaction, nested polymerase chain reaction, ligase chain reaction (Barany F. (1991) PNAS 88:189-193; Barany F. (1991) PCR Methods and Applications 1:5-16), ligase detection reaction (Barany F. (1991) PNAS 88:189-193), strand displacement amplification, transcription based amplification system, nucleic acid sequence-based amplification, rolling circle amplification, and hyper-branched rolling circle amplification.

In certain embodiments, the amplification reaction is the polymerase chain reaction. Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target sequence includes introducing an excess of oligonucleotide primers to a DNA mixture containing a desired target sequence, followed by a precise sequence of thermal cycling in the presence of a DNA polymerase. Primer sets introduced to the droplets can include primers specific to reference target, one or more different methylation-dependent targets (e.g. target methylation-dependent CpG regions), and control nucleic acid molecules (e.g. specific to the cleavage site of the control nucleic acid molecules).

To effect amplification, primers are annealed to their complementary sequence within the target molecule. Following annealing, the primers are extended with a polymerase so as to form a new complementary strand. The steps of denaturation, primer annealing and polymerase extension can be repeated many times (i.e., denaturation, annealing and extension constitute one cycle; there can be numerous cycles) to obtain a high concentration of an amplified segment of a desired target sequence. The length of the amplified segment of the desired target sequence is determined by relative positions of the primers with respect to each other, and therefore, this length is a controllable parameter.

Methods for performing PCR in droplets are shown for example in Link et al. (U.S. patent application numbers 2008/0014589, 2008/0003142, and 2010/0137163), Anderson et al. (U.S. Pat. No. 7,041,481 and which reissued as RE41,780) and European publication number EP2047910 to Raindance Technologies Inc. The content of each of which is incorporated by reference herein in its entirety.

The sample droplet may be pre-mixed with a primer or primers, or the primer or primers may be added to the droplet. In some embodiments, droplets created by segmenting the starting sample are merged with a second set of droplets including one or more primers for the target nucleic acid in order to produce final droplets. The merging of droplets can be accomplished using, for example, one or more droplet merging techniques described for example in Link et al. (U.S. patent application numbers 2008/0014589, 2008/0003142, and 2010/0137163) and European publication number EP2047910 to Raindance Technologies Inc.

In embodiments involving merging of droplets, two droplet formation modules are used. In one embodiment, a first droplet formation module produces the sample droplets consistent with limiting or terminal dilution of reference target nucleic acid, methylation-dependent nucleic acid, and control nucleic acid. A second droplet formation or reinjection module inserts droplets that contain reagents for a PCR reaction. Such droplets generally include the “PCR master mix” (known to those in the art as a mixture containing at least Taq polymerase, deoxynucleotides of type A, C, G and T, and magnesium chloride) and forward and reverse primers (known to those in the art collectively as “primers”), all suspended within an aqueous buffer. The second droplet also includes detectably labeled probes for detection of the amplified target nucleic acids (whether the reference targets, control, methylation-dependent target), the details of which are discussed below. Different arrangements of reagents between the two droplet types is envisioned. For example, in another embodiment, the template droplets also contain the PCR master mix, but the primers and probes remain in the second droplets. Any arrangement of reagents and template DNA can be used according to the invention.

Primers can be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers can also be obtained from commercial sources such as Operon Technologies, Amersham Pharmacia Biotech, Sigma, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and, length of the primer pairs yield the desired melting temperature. The simplest equation for determining the melting temperature of primers smaller than 25 base pairs is the Wallace Rule (Td=2(A+T)+4(G+C)). Another method for determining the melting temperature of primers is the nearest neighbor method Computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The T_{M} (melting or annealing temperature) of each primer is calculated using software programs such as Oligo Design, available from Invitrogen Corp.

In one embodiment, the droplet formation modules are arranged and controlled to produce an interdigitation of sample droplets and PCR reagent droplets flowing through a channel. Such an arrangement is described for example in Link et al. (U.S. patent application numbers 2008/0014589, 2008/0003142, and 2010/0137163) and European publication number EP2047910 to Raindance Technologies Inc.

A sample droplet is then caused to merge with a PCR reagent droplet, producing a droplet that includes the PCR master mix, primers, detectably labeled probes, and the target nucleic acid. Droplets may be merged for example by: producing dielectrophoretic forces on the droplets using electric field gradients and then controlling the forces to cause the droplets to merge; producing droplets of different sizes that thus travel at different velocities, which causes the droplets to merge; and producing droplets having different viscosities that thus travel at different velocities, which causes the droplets to merge with each other. Each of those techniques is further described in Link et al. (U.S. patent application numbers 2008/0014589, 2008/0003142, and 2010/0137163) and European publication number EP2047910 to Raindance Technologies Inc. Further description of producing and controlling dielectrophoretic forces on droplets to cause the droplets to merge is described in Link et al. (U.S. patent application number 2007/0003442) and European Patent Number EP2004316 to Raindance Technologies Inc.

In another embodiment, called simple droplet generation, a single droplet formation module, or a plurality of droplet formation modules are arranged to produce droplets from a mixture already containing the template DNA, the PCR master mix, primers, and detectably labeled probes. In yet another embodiment, called co-flow, upstream from a single droplet formation module two channels intersect allowing two flow streams to converge. One flow stream contains one set of reagents and the template DNA, and the other contains the remaining reagents. In the preferred embodiment for co-flow, the template DNA and the PCR master mix are in one flow stream, and the primers and probes are in the other. However, the invention is not limited in regard to the constituency of either flow stream. For example, in another embodiment, one flow stream contains just the template DNA, and the other contains the PCR master mix, the primers, and the probes. On convergence of the flow streams in a fluidic intersection, the flow streams may or may not mix before the droplet generation nozzle. In either embodiment, some amount of fluid from the first stream, and some amount of fluid from the second stream are encapsulated within a single droplet. Following encapsulation, complete mixing occurs.

Once final droplets have been produced by any of the droplet forming embodiments above, or by any other embodiments, the droplets are thermal cycled, resulting in amplification of the target nucleic acid in each droplet. In certain embodiments, the droplets are collected off-chip as an emulsion in a PCR thermal cycling tube and then thermally cycled in a conventional thermal cycler. Temperature profiles for thermal cycling can be adjusted and optimized as with any conventional DNA amplification by PCR.

In certain embodiments, the droplets are flowed through a channel in a serpentine path between heating and cooling lines to amplify the nucleic acid in the droplet. The width and depth of the channel may be adjusted to set the residence time at each temperature, which can be controlled to anywhere between less than a second and minutes.

In certain embodiments, the three temperature zones are used for the amplification reaction. The three temperature zones are controlled to result in denaturation of double stranded nucleic acid (high temperature zone), annealing of primers (low temperature zones), and amplification of single stranded nucleic acid to produce double stranded nucleic acids (intermediate temperature zones). The temperatures within these zones fall within ranges well known in the art for conducting PCR reactions. See for example, Sambrook et al. (Molecular Cloning, A Laboratory Manual, 3^{rd} edition, Cold Spring Harbor Laboratory Press, Cold Spring Harbor, New York, 2001).

In certain embodiments, the three temperature zones are controlled to have temperatures as follows: 95° C. (T_{H}), 55° C. (T_{L}), 72° C. (T_{M}). The prepared sample droplets flow through the channel at a controlled rate. The sample droplets first pass the initial denaturation zone (T_{H}) before thermal cycling. The initial preheat is an extended zone to ensure that nucleic acids within the sample droplet have denatured successfully before thermal cycling. The requirement for a preheat zone and the length of denaturation time required is dependent on the chemistry being used in the reaction. The samples pass into the high temperature zone, of approximately 95° C., where the sample is first separated into single stranded DNA in a process called denaturation. The sample then flows to the low temperature, of approximately 55° C., where the hybridization process takes place, during which the primers anneal to the complementary sequences of the sample. Finally, as the sample flows through the third medium temperature, of approximately 72° C., the polymerase process occurs when the primers are extended along the single strand of DNA with a thermostable enzyme. Methods for controlling the temperature in each zone may include but are not limited to electrical resistance, Peltier junction, microwave radiation, and illumination with infrared radiation.

The nucleic acids undergo the same thermal cycling and chemical reaction as the droplets passes through each thermal cycle as they flow through the channel. The total number of cycles in the device is easily altered by an extension of thermal zones or by the creation of a continuous loop structure. The sample undergoes the same thermal cycling and chemical reaction as it passes through N amplification cycles of the complete thermal device.

In other embodiments, the temperature zones are controlled to achieve two individual temperature zones for a PCR reaction. In certain embodiments, the two temperature zones are controlled to have temperatures as follows: 95° C. (T_{H}) and 60° C. (T_{L}). The sample droplet optionally flows through an initial preheat zone before entering thermal cycling. The preheat zone may be important for some chemistry for activation and also to ensure that double stranded nucleic acid in the droplets are fully denatured before the thermal cycling reaction begins. In an exemplary embodiment, the preheat dwell length results in approximately 10 minutes preheat of the droplets at the higher temperature.

The sample droplet continues into the high temperature zone, of approximately 95° C., where the sample is first separated into single stranded DNA in a process called denaturation. The sample then flows through the device to the low temperature zone, of approximately 60° C., where the hybridization process takes place, during which the primers anneal to the complementary sequences of the sample. Finally the polymerase process occurs when the primers are extended along the single strand of DNA with a thermostable enzyme. The sample undergoes the same thermal cycling and chemical reaction as it passes through each thermal cycle of the complete device. The total number of cycles in the device is easily altered by an extension of block length and tubing.

In another embodiment the droplets are created and/or merged on chip followed by their storage either on the same chip or another chip or off chip in some type of storage vessel such as a PCR tube. The chip or storage vessel containing the droplets is then cycled in its entirety to achieve the desired PCR heating and cooling cycles.

In another embodiment the droplets are collected in a chamber where the density difference between the droplets and the surrounding oil allows for the oil to be rapidly exchanged without removing the droplets. The temperature of the droplets can then be rapidly changed by exchange of the oil in the vessel for oil of a different temperature. This technique is broadly useful with two and three step temperature cycling or any other sequence of temperatures.

The invention is not limited by the method used to thermocycle the droplets. Any method of thermocycling the droplets may be used.

### Target Detection

After amplification, droplets are flowed to a detection module for detection of amplification products. For embodiments in which the droplets are thermally cycled off-chip, the droplets require re-injection into either a second fluidic circuit for read-out—that may or may not reside on the same chip as the fluidic circuit or circuits for droplet generation—or in certain embodiments the droplets may be reinjected for read-out back into the original fluidic circuit used for droplet generation. The droplets may be individually analyzed and detected using any methods known in the art, such as detecting the presence or amount of a reporter. Generally, the detection module is in communication with one or more detection apparatuses. The detection apparatuses can be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module. Further description of detection modules and methods of detecting amplification products in droplets are shown in Link et al. (U.S. patent application numbers 2008/0014589, 2008/0003142, and 2010/0137163) and European publication number EP2047910 to Raindance Technologies Inc.

In certain embodiments, amplified target (control, one or more different reference targets, and one or more different methylation-dependent targets) are detected using detectably labeled probes. In particular embodiments, the detectably labeled probes are optically labeled probes, such as fluorescently labeled probes. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are FAM and VIC™ (from Applied Biosystems). Labels other than fluorescent labels are contemplated by the invention, including other optically-detectable labels.

In certain aspects, the droplets of the invention contain a plurality of detectable probes that hybridize to amplicons produced in the droplets. Members of the plurality of probes can each include the same detectable label, or a different detectable label. The plurality of probes can also include one or more groups of probes at varying concentration. The groups of probes at varying concentrations can include the same detectable label which vary in intensity, due to varying probe concentrations.

In some embodiments, the droplets of the invention contain a plurality of barcodes that hybridize to amplicons produced in the droplets or are incorporated into the amplicons. The barcodes may be used in lieu of fluorescent probes, to detect the presence of a target sequence, or the barcodes can be used in addition to fluorescent probes, to track a multitude of sample sources. A detectable barcode-type label can be any barcode-type label known in the art including, for example, barcoded magnetic beads (e.g., from Applied Biocode, Inc., Santa Fe Springs, CA), and nucleic acid sequences. Nucleic acid barcode sequences typically include a set of oligonucleotides ranging from about 4 to about 20 oligonucleotide bases (e.g., 8-10 oligonucleotide bases) and uniquely encode a discrete library member without containing significant homology to any sequence in the targeted sample.

The barcode sequence generally includes features useful in sequencing reactions. For example, the barcode sequences are designed to have minimal or no homopolymer regions, i.e., 2 or more of the same base in a row such as AA or CCC, within the barcode sequence. The barcode sequences are also designed so that they are at least one edit distance away from the base addition order when performing base-by-base sequencing, ensuring that the first and last base do not match the expected bases of the sequence. In certain embodiments, the barcode sequences are designed to be correlated to a particular subject, allowing subject samples to be distinguished. Designing barcodes is shown U.S. Pat. No. 6,235,475, the contents of which are incorporated by reference herein in their entirety.

In some instances, the primers used in the invention may include barcodes such that the barcodes will be incorporated into the amplified products. For example, the unique barcode sequence could be incorporated into the 5′ end of the primer, or the barcode sequence could be incorporated into the 3′ end of the primer. In some embodiments, the barcodes may be incorporated into the amplified products after amplification. For example, a suitable restriction enzyme (or other endonuclease) may be introduced to a sample, e.g., a droplet, where it will cut off an end of an amplification product so that a barcode can be added with a ligase. Attaching barcode sequences to nucleic acids is shown in U.S. Pub. 2008/0081330 and PCT/US09/64001, the content of each of which is incorporated by reference herein in its entirety. Methods for designing sets of barcode sequences and other methods for attaching barcode sequences are shown in U.S. Pat. Nos. 6,138,077; 6,352,828; 5,636,400; 6,172,214; 6235,475; 7,393,665; 7,544,473; 5,846,719; 5,695,934; 5,604,097; 6,150,516; RE39,793; 7,537,897; 6,172,218; and 5,863,722, the content of each of which is incorporated by reference herein in its entirety. For sequencing detection, the amplified contents of the droplets may be released for multiplex sequencing.

In a separate embodiment the detection can occur by the scanning of droplets confined to a monolayer in a storage device that is transparent to the wavelengths or method or detection. Droplets stored in this fashion can be scanned either by the movement of the storage device by the scanner or the movement of the scanner over the storage device.

The invention is not limited to the TaqMan assay, as described above, but rather the invention encompasses the use of all fluorogenic DNA hybridization probes, such as molecular beacons, Solaris probes, scorpion probes, and any other probes that function by sequence specific recognition of target DNA by hybridization and result in increased fluorescence on amplification of the target sequence.

### Normalization

According to methods of the invention, the quantification of methylation-dependent targets in the assay can be normalized based on the efficiency of the methylation state specific enzymatic reaction. For example, the quantified results of the assay are normalized, after detection, based on a number of unreacted control molecules that had been spiked-in at a known number of molecules per μl of sample and are remain in the sample relative to the number of control molecules introduced into the sample (i.e. measured efficiency/completeness of the enzymatic reaction). In certain embodiments, a number of the detected methylation state specific targets are normalized based on the measured efficiency. In certain embodiments, a ratio of the detected methylation-dependent targets to the reference targets is determined, and then the ratio is normalized based on the measured efficiency.

### Digital PCR Performance in Droplets

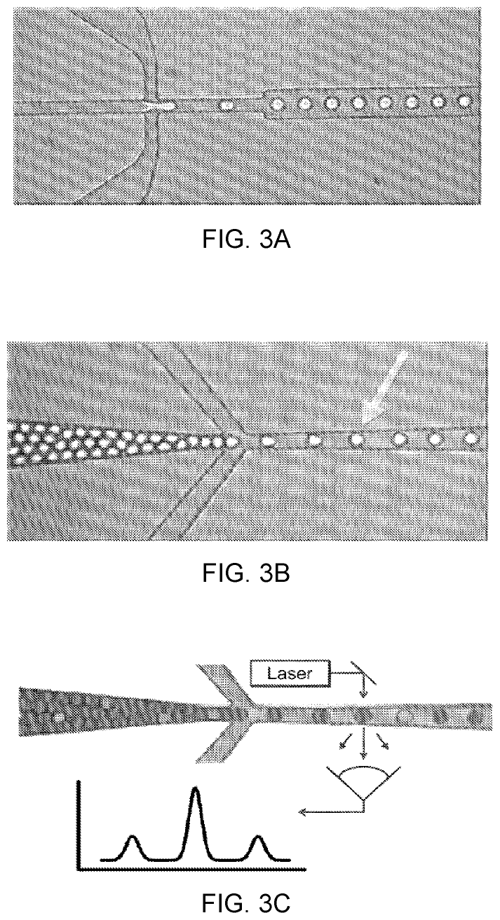

An exemplary microfluidic system for droplet generation and readout is depicted in FIG. 3. The microfluidic system is capable of both droplet generation and readout. As shown in FIG. 3A (droplet generation chip), a continuous aqueous phase containing the PCR master mix, primers, and probes, and template DNA flows into the fluidic intersection from the left, and the carrier oil enters from the top and bottom. An emerging bolus of aqueous liquid is imaged inside the intersection just prior to snapping off into a discrete 4 pL droplet as the fluidic strain begins to exceed the surface tension of the aqueous liquid. The steady train of droplets leaving the intersection toward the right is collected off chip as a stable emulsion for thermal cycling. FIG. 3B depicts the droplet spacing for readout. Flows are arranged as in 3A, except instead of a continuous phase, the emulsion from (A) is injected from the left into the intersection after thermal cycling. The oil is drained from the emulsion during off-chip handling, hence the emulsion appears tightly packed in the image before the intersection. The oil introduced in the intersection separates the droplets and the fluorescence of each droplet is measured at the location marked by the arrow. FIG. 3C depicts a cartoon of droplet readout by fluorescence. The relatively infrequent PCR(+) droplets (light gray) flow along with the majority of PCR(−) droplets (dark gray) toward the detector. The droplets are interrogated sequentially by laser induced fluorescence while passing through the detection region.

In a serial dilution, the average number of target DNA molecules per droplet—called the “occupancy” from this point forward—decreases in direct proportion to the DNA concentration. The occupancy is calculated from Poisson statistics using the following equation well known to those experienced in the art:

$$occupancy = ln ( \frac{P + N}{N} ) , \qquad ( 1 )$$

where P and N are the numbers of PCR(+) and PCR(−) droplets respectively.

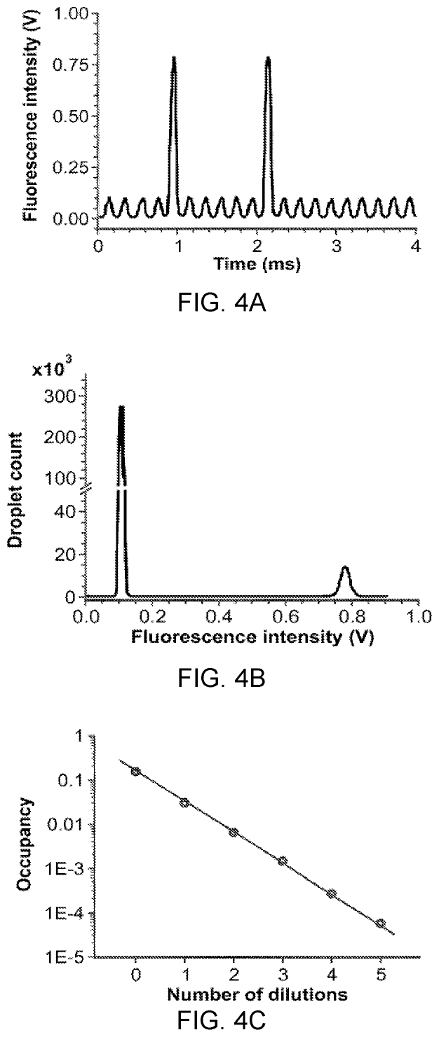

Droplets are analyzed by fluorescence while flowing through the readout chip to count the numbers of PCR(+) and PCR(−) droplets (see FIG. 3C). As each droplet passes the detection zone (marked with an arrow in FIG. 3B), a burst of fluorescence is observed. To account for small run-to-run differences in the fluorescence intensity that can occur due to different chip positioning, etc., each set of data is scaled such that the average fluorescence intensity of the empty droplets is 0.1 V. FIG. 4A shows a very short duration of a typical trace of fluorescence bursts from individual droplets for the sample with the highest DNA concentration in the series. PCR(+) and PCR(−) droplets are easily discriminated by fluorescence intensity. The two large bursts of fluorescence peaking at ˜0.8 V arise from the PCR(+) droplets, whereas smaller bursts, due to incomplete fluorescence quenching in the PCR(−) droplets, peak at ˜0.1 V. A histogram of peak intensities from the complete data set reveals two clear populations centered at 0.10 and 0.78 V (FIG. 4B), demonstrating that the trend evident in the short trace in FIG. 4A is stable over much longer periods of time. Integration over the two populations in FIG. 4B yields a total of 197,507 PCR(+) and 1,240,126 PCR(−) droplets. Hence the occupancy was 0.15 for this sample by Eqn. 1, corresponding to the expected occupancy of 0.18 based on the measured DNA concentration of 110 ng/μL. The occupancy is measured for each sample in the serial dilution and fit to the dilution equation:

$$occupancy ( n ) = \frac{A}{{f}^{n}} , \qquad ( 2 )$$

where n is the number of dilutions, A is the occupancy at the starting concentration (n=0), and ƒ is the dilution factor. The linear fit was in excellent agreement with the data, with an R² value of 0.9999 and the fitted dilution factor of 4.8 in close agreement with the expected value of 5.0.

### Copy Number Assay

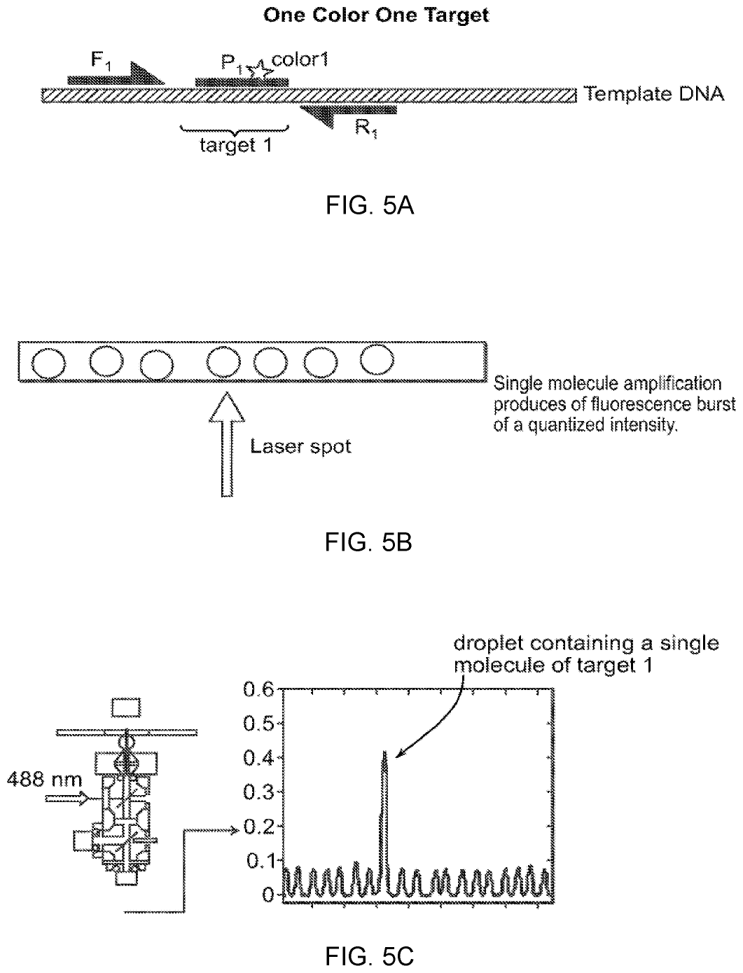

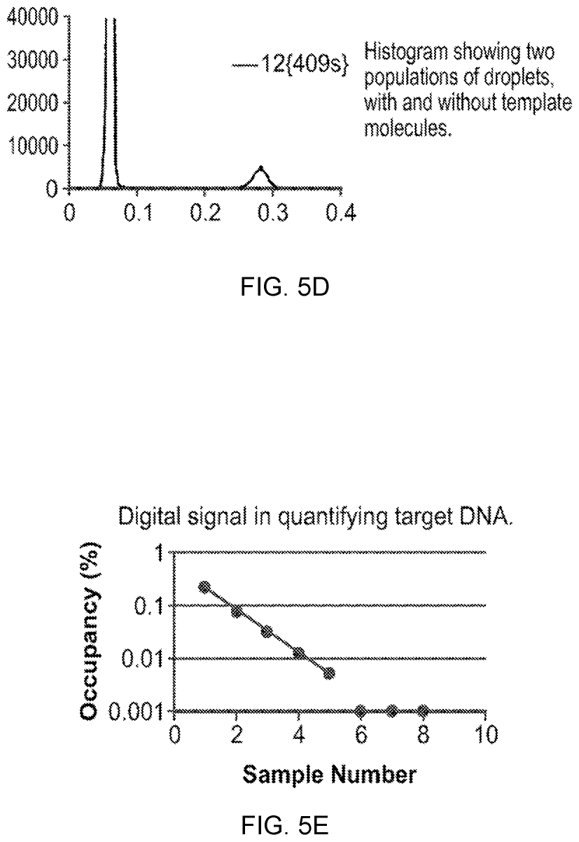

Traditional digital PCR methods involve the use of a single labeled probe specific for an individual target. FIG. 5 is a schematic depicting one-color detection of a target dsDNA sequence using droplet based digital PCR. As shown in Panel A of FIG. 5, a template DNA is amplified with a forward primer (F1) and a reverse primer (R1). Probe (P1) labeled with a fluorophore of color 1 binds to the target genetic sequence (target 1). Microdroplets are made of diluted solution of template DNA under conditions of limiting or terminal dilution. Droplets containing the target sequence emit fluorescence and are detected by laser (Panels B and C). The number of microcapsules either containing or not containing the target sequence is shown in a histogram (D) and quantified (E).

### Data Analysis

Analysis is then performed on the droplets. The analysis may be based on counting, i.e., i.e., determining a number of droplets containing a methylation-independent target, determining a number of droplets containing a methylated-dependent target. In some embodiments the first and second numbers are analyzed to determine whether a statistical difference exists, in other embodiments the first and second numbers are analyzed to determine a ratio with different values for the numerator/denominator compared to that expected in a ‘normal’ sample.

It will be appreciated that various counting schemes useful with embodiments of the invention can include: 1) ratios of targets located on the same contiguous molecule (e.g. chromosome, or chromosome segment); 2) ratio on different contiguous molecule (fetal case looks at chr21 vs. chr18 vs. ‘normal diploid’ chromosome); 3) ratios between ‘control’ targets for multiple measurements of enzymatic specificity or completion and additional input target metrics. 4) Imbalances measure amplification/deletion/enrichment/depletion of the targets.

Counting methods are well known in the art. See, e.g., Lapidus et al. (U.S. Pat. Nos. 5,670,325 and 5,928,870) and Shuber et al. (U.S. Pat. Nos. 6,203,993 and 6,214,558), the content of each of which is incorporated by reference herein in its entirety. Statistical difference may be indicative of a condition. In certain embodiments, the condition is a disease such as cancer. In other embodiments, the statistical different relates to fetal aneuploidy. In other embodiments, the difference between a number of reference targets and a number of methylated-dependent targets provides for quantification of an amount of fetal nucleic acid in maternal blood.

It is important to note that for the result to be highly quantitative, the “methylation-specific” target(s) should show the specific target-state with a high degree of penetrance. For example, for detection of fetal aneuploidy, the minor ‘fetal’ target should be 100% methylated/unreactable state for all fetal-derived target molecules, with the maternal targets showing the complete unmethylated/reactable state (e.g. 0% methylated). It will be appreciated by those of ordinary skill in the art that appropriate validation of the targets is important, and that embodiments of the invention are useful as validation process itself (i.e. one ‘validated’ target can be compared to a second ‘candidate’ target).

Numerous examples of methylated genes that have been linked to various types of cancer have been identified. Examples of methylated genes that have been linked with susceptibility to or incidence of colorectal cancer include, for example, FOXE1, SOX17, SYNE1, BOLL, CABYR, EFEMP1, FBLN2, FOXL2, GNB4, GSTM3, HoxD1, Jph3, Neuralized (NEURL), PPP1R14a, TP53AP1, RAB32, APC2, GPNMB, MMP2, EVL, STARD8, PTPRD, CD109, LGR6, RET, CHD5, RNF182, ICAM5, ARMCX2, CBR1, DDX43, DMRTB1, FBLN2, HIST2H2AA, ICAM1, LY6K, NEF3, POMC, STK31, SYCP3, TCL1A, TFPI-2, TLR2, UCHL1, ZFP42, ASCL2, ATP8A2, CTAG2, EPHA4, FANCF, FOXQ1, HUS1B, JAM3, LEF1, MOV10L1, NPPB, PWWP1, RASSF5, REC8L1, SALL4, BEX1, BNIP3, CCK, CDX1, CNN3, CXX1, IRX4, MC5R, RSNL2, SMARCA3, SPON1, SYT6, TRPC3, TSPYL6, ZNF345, DKK3, ZNF655, B4GALT1, C10orf119, C10orf13, CBR1, COPS4, COVA1, CSRP1, DARS, DNAJC10, FKBP14, FN3KRP, GANAB, HUS1, KLF11, MRPL4, MYLK, NELF, NETO2, PAPSS2, RBMS2, RHOB, SECTM1, SIRT2, SIRT7, SLC35D1, SLC9A3R1, TTRAP, TUBG2, FLJ20277, MYBL2, GPR116, QSMR, PC4, SLC39A4, UBE3A, PDLIM3, UBE21, or any combination thereof.

Examples of methylated genes that have been linked with susceptibility to or incidence of prostate cancer include, for example, GSTP1, APC, PTGS2, T1G1, EDNRB, RASS1a, GSTP1, APC, PTGS2, T1G1, EDNRB, CD3D, APOC1, NBL1, ING4, LEF1, CENTD3, MGC15396, FKBP4, PLTP, TFAP2A, ATXN1, BMP2, ENPEP, MCAM, SSBP2, PDLIM3, NDP, or any combination thereof.

Examples of methylated genes that have been linked with susceptibility to or incidence of breast cancer include, for example, PITX2, PITX2, BACH1, CKMT, GALE, HMG20B, KRT14, OGDHL, PON2, SESN1, KIF1A (kinesin family member 1A) PDLIM3, MAL (T cell proliferation protein), or any combination thereof.

Examples of methylated genes that have been linked with susceptibility to or incidence of lung cancer include, for example, p16INK4a, APC, TMS1, RASSF1, DAPK, PRSS3 (serine protease family member-trypsinogen IV—a putative tumor suppressor gene), human DAB2 interactive protein gene, apoptosis-associated speck-lick protein containing a CARD, p16, FHIT, H-cadherin, RARβ, RARB2, PHKA2, CBR3, CAMK4, HOXB5, ZNF198, RGS4, RBM15B, PDLIM3, PAK3, PIGH, TUBB4, NISCH or any combination thereof.

Examples of methylated markers that have been shown to be associated with susceptibility to or incidence of gastrointestinal cancer include, without limitation, NDRG4/NDRG2 subfamily gene, GATA4, OSMR, GATA5, SFRP1, ADAM23, JPH3, SFRP2, APC, MGMT, TFPI2, BNIP3, FOXE1, SYNE1, SOX17, PHACTR3, JAM3, or any combination thereof.

Examples of methylated genes that have been linked with susceptibility to or incidence of cervical cancer include, for example, ESR1, DAP-kinase, APC, TIMP-3, RAR-beta, CALCA, TSLC1, TIMP-2, DcR1, DcR2, BRCA1, p15, Rassf1A, MLH1, MGMT, PDCD4, TFPI2, ARMC7, TRM-HUMAN, OGDHL, PTGS2, CDK6, GPR39, HMGN2, C130RF18, ASMTL, DLL4, NP-659450.1, NP-078820.1, CLU, HPCA, PLCG2, RALY, GNB4, CCNA1, NPTX1, C90RF19, or any combination thereof.

Examples of “methylation-specific” genes that have been linked with fetal conditions include RASSF1/MASPIN/others. It will also be appreciated that un-methylated markers are also useful with embodiments of the invention.

### Release of Target from Droplet

Methods of the invention may further involve releasing amplified target molecules from the droplets for further analysis. Methods of releasing amplified target molecules from the droplets are shown in for example in Link et al. (U.S. patent application numbers 2008/0014589, 2008/0003142, and 2010/0137163) and European publication number EP2047910 to RainDance Technologies Inc.

In certain embodiments, sample droplets are allowed to cream to the top of the carrier fluid. By way of non-limiting example, the carrier fluid can include a perfluorocarbon oil that can have one or more stabilizing surfactants. The droplet rises to the top or separates from the carrier fluid by virtue of the density of the carrier fluid being greater than that of the aqueous phase that makes up the droplet. For example, the perfluorocarbon oil used in one embodiment of the methods of the invention is 1.8, compared to the density of the aqueous phase of the droplet, which is 1.0.

The creamed liquids are then placed onto a second carrier fluid which contains a de-stabilizing surfactant, such as a perfluorinated alcohol (e.g. 1H,1H,2H,2H-Perfluoro-1-octanol). The second carrier fluid can also be a perfluorocarbon oil. Upon mixing, the aqueous droplets begins to coalesce, and coalescence is completed by brief centrifugation at low speed (e.g., 1 minute at 2000 rpm in a microcentrifuge). The coalesced aqueous phase can now be removed and the further analyzed.

The released amplified material can also be subjected to further amplification by the use tailed primers and secondary PCR primers. In this embodiment the primers in the droplet contain an additional sequence or tail added onto the 5′ end of the sequence specific portion of the primer. The sequences for the tailed regions are the same for each primer pair and are incorporated onto the 5′ portion of the amplicons during PCR cycling. Once the amplicons are removed from the droplets, another set of PCR primers that can hybridize to the tail regions of the amplicons can be used to amplify the products through additional rounds of PCR. The secondary primers can exactly match the tailed region in length and sequence or can themselves contain additional sequence at the 5′ ends of the tail portion of the primer. During the secondary PCR cycling these additional regions also become incorporated into the amplicons. These additional sequences can include, but are not limited to adaptor regions utilized by sequencing platforms for library preparation and sequencing, sequences used as a barcoding function for the identification of samples multiplexed into the same reaction. molecules for the separation of amplicons from the rest of the reaction materials such as biotin, digoxin, peptides, or antibodies and molecules such as fluorescent markers that can be used to identify the fragments.

In certain embodiments, the amplified target molecules are sequenced. In a particular embodiment, the sequencing is single-molecule sequencing-by-synthesis. Single-molecule sequencing is shown for example in Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslavsky, et al., PNAS (USA), 100: 3960-3964 (2003), the contents of each of these references is incorporated by reference herein in its entirety.

Briefly, a single-stranded nucleic acid (e.g., DNA or cDNA) is hybridized to oligonucleotides attached to a surface of a flow cell. The single-stranded nucleic acids may be captured by methods known in the art, such as those shown in Lapidus (U.S. Pat. No. 7,666,593). The oligonucleotides may be covalently attached to the surface or various attachments other than covalent linking as known to those of ordinary skill in the art may be employed. Moreover, the attachment may be indirect, e.g., via the polymerases of the invention directly or indirectly attached to the surface. The surface may be planar or otherwise, and/or may be porous or non-porous, or any other type of surface known to those of ordinary skill to be suitable for attachment. The nucleic acid is then sequenced by imaging the polymerase-mediated addition of fluorescently-labeled nucleotides incorporated into the growing strand surface oligonucleotide, at single molecule resolution.

### Quantification of Fetal Nucleic Acid

Methods of the invention are useful for quantifying fetal nucleic acid in maternal blood in order to perform non-invasive tests for fetal genomic abnormalities. Such methods involve obtaining a sample, e.g., a tissue or body fluid that is suspected to include both maternal and fetal nucleic acids. Such samples may include urine, vaginal secretion, amniotic fluid, or tissue. In certain embodiments, this sample is drawn maternal blood, and circulating DNA is found in the blood plasma, rather than in cells. A preferred sample is maternal peripheral venous blood.

Because the amount of fetal nucleic acid in a maternal sample generally increases as a pregnancy progresses, less sample may be required as the pregnancy progresses in order to obtain the same or similar amount of fetal nucleic acid from a sample.

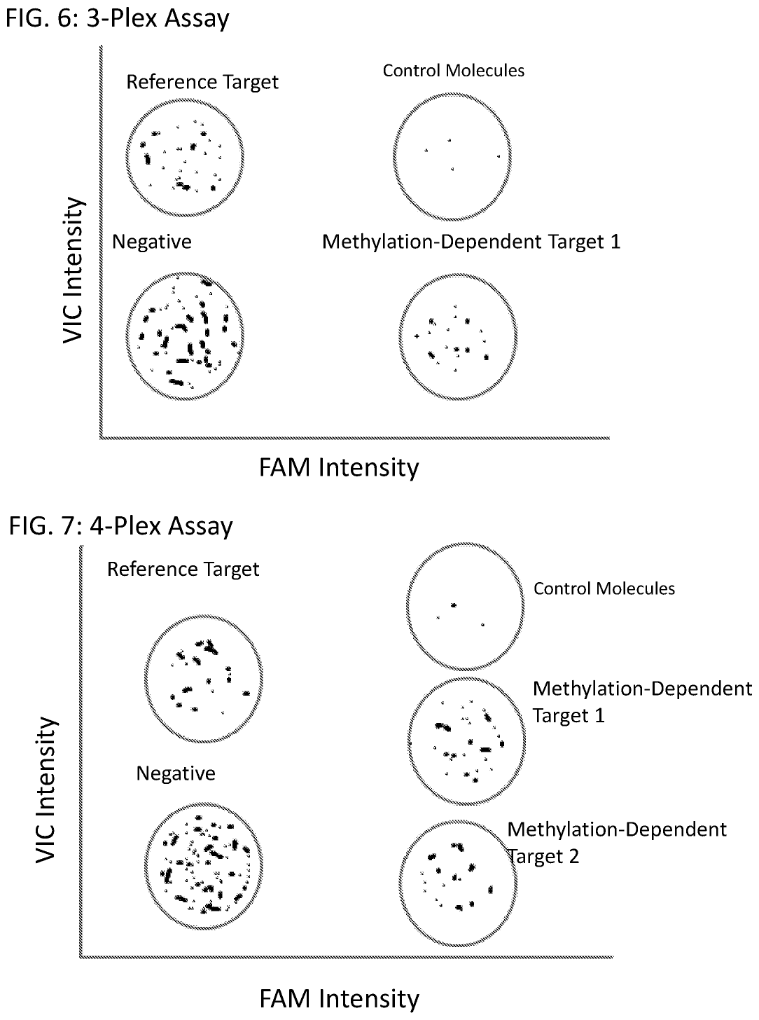

FIG. 6 illustrates expected results of a digital PCR 3-plex assay used to determine the percent of nucleic acid molecules in a sample deriving from the fetus in maternal blood. Specifically, the assay illustrates the ability to determine the percent of fetal nucleic acid when the methylation state specific target is a locus where the copy number is known (e.g. a normal diploid cell/patient typically has 2 copies of each gene) and is unlikely to exhibit copy number variation, and the methylation state in the fetus is different than the maternal methylation state that enables the calculation of a ratio between the two states (e.g. promoter of RASSF1A gene on chromosome 3).

FIG. 6 shows a typical dPCR cluster plot (VIC intensity on the y-axis; FAM intensity on the x-axis) with circular gates used to count the number of droplets containing certain content. As shown in FIG. 6, the bottom-left circular gate shows the number of droplets containing no target molecules (Negative). The top-left circular gate shows the number of droplets containing a reference target (Methylation state specific targets or MSRE enzymatic reaction-independent). The reference molecule is not a substrate for enzymatic reaction by the MSRE's and used to quantify the total number of amplifiable genome-equivalents in the sample. The bottom-right circular gate shows a number of droplets containing a methylation-enzymatic reaction-dependent target. The methylation state specific target is used to quantify the fetal-specific methylated nucleic acid in the sample. The top-right circular gate shows the number of control nucleic acid molecules remaining after enzymatic reaction. The unreacted control nucleic acid molecules are used to quantify the completion of a methylation state specific enzymatic reaction, and a known quantity of control molecules was added to the sample prior to methylation state specific enzymatic reaction. The number of unreacted control molecules compared to the known quantity of control molecules is used to measure the efficiency of the enzymatic reaction (e.g. percent efficiency). The number of methylation state specific targets is normalized based on that measured efficiency. The ratio of the adjusted number of methylation state specific target molecules to the number of reference molecules provides the percent of the sample nucleic acid that derives from the fetus.

In addition to determining the percent of sample nucleic acid that derives from the fetus, a similar 3-plex assay can be used to directly quantify and score a sample for a fetal aneuploidy if the methylation specific target is located on the aneuploid chromosome and the reference target molecule is on a diploid chromosome (e.g. where the ratio is different from 1:1). For example the percent fetal fraction (or tumor in the case of cancer) matters for the precision of the assay and may not be known a priori without performing an assay according to the embodiments described herein. Fetal aneuploidy (e.g., Down syndrome, Edward syndrome, and Patau syndrome) and other chromosomal aberrations affect 9 of 1,000 live births (Cunningham et al. in Williams Obstetrics, McGraw-Hill, New York, p. 942, 2002). Chromosomal abnormalities are generally diagnosed by karyotyping of fetal cells obtained by invasive procedures such as chorionic villus sampling or amniocentesis. Those procedures are associated with potentially significant risks to both the fetus and the mother. Noninvasive screening using maternal serum markers or ultrasound are available but have limited reliability (Fan et al., PNAS, 105 (42):16266-16271, 2008).

Methods of the invention may be used to screen for fetal aneuploidy; for example, if the methylation state specific target is on a chromosome with potential trisomy (a type of aneuploidy), and the reference target is on a different chromosome that does not have trisomy. In such an example, a statistically-signification deviation from a 1:1 ratio in the number of droplets containing a methylation state specific target molecule and the number of droplets containing a reference molecule is indicative of trisomy. In such embodiment, the methylation state specific target is a target on chromosome 21 that has been validated as always and only being methylated in fetal nucleic acid, and the reference molecule is a target on a chromosome without copy number variation. In certain embodiments, the aneuploidy is trisomy of chromosome 21 (Down syndrome). In other embodiments, the aneuploidy may include trisomy of chromosomes 13 and 18.

As illustrated in FIG. 7, methods of the invention incorporate more than one methylation state specific target, reference molecule, and control nucleic acid in order to conduct multiple determinations in a single assay. As shown in FIG. 7, the typical dPCR cluster plot includes circular gates of droplets containing a first methylation state specific target molecule and droplets containing a second methylation state specific target molecule. This concept may be expanded further include multiple different methylation state specific target molecules and multiple different reference molecules. Referring back to FIG. 7, a count of droplets containing the first-methylation state specific target molecules can be used to determine a percentage fetal nucleic acid derived from the sample (as described above), and the second methylation state specific target molecules can be used to quantify and score the sample for a potential aneuploidy (as described above).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

1-20. (canceled)

21. A method for analysis of nucleic acid, the method comprising:
obtaining a sample comprising (i) target nucleic acid comprising a locus and (ii) non-target nucleic acid comprising the locus, wherein the one of the (i) target nucleic acids and (ii) the non-target nucleic acid is methylated and the other is unmethylated;
treating a sample with a methylation-specific restriction enzyme (MSRE) to selectively digest only the non-target nucleic acid;
partitioning the sample into compartments;
performing amplification in the compartments with detectably-labeled probes specific to the locus; and
detecting signal from the probes to determine a quantity of the target nucleic acids that was present in the sample.

22. The method of claim 21, wherein the target nucleic acid comprises fetal nucleic acid and the sample comprises maternal blood or plasma.

23. The method of claim 22, wherein the target nucleic acid comprises maternal nucleic acid.

24. The method of claim 21, further comprising: measuring by digital PCR a quantity of a reference molecule in the sample that is not digested by the MSRE; and quantifying the target nucleic acid by comparison to the measured reference molecule.

25. The method of claim 21, further comprising adding into the sample a known quantity of a reference molecule that contains the locus and is not digested by the MSRE; measuring by digital PCR the reference molecule in the sample, and quantifying the target nucleic acid by comparison to the measured reference molecule.

26. The method of claim 25, wherein the target nucleic acid comprises fetal nucleic acid.

27. The method of claim 21, further comprising scoring the sample for a fetal aneuploidy.

28. The method of claim 21, wherein the detectably labeled probes comprise hydrolysis probes or molecular beacons.

29. The method of claim 21, wherein each of the detectably labeled probes is labeled with a fluorophore selected from the group consisting of: VIC, FAM, an Atto dye, a cyanine dye, ROX, Cy5, Cy5.5, malachite green, and Texas red.

30. The method of claim 21, further comprising reporting a condition in a subject based on the determined quantity of the nucleic acids in the first modification state in the sample.

31. The method of claim 21, further comprising performing the method with a known quantity of a control molecule that contains the locus and is not digested by the MSRE; measuring a quantity of the control molecule by digital PCR present after the treating step; and using the measured quantity of the control molecule to quantify completion of the selective digestion of the non-target nucleic acid.

32. The method of claim 21, wherein the compartments are aqueous droplets.

33. The method of claim 32, wherein each droplet is surrounded by an oil comprising a surfactant.

34. The method of claim 21, wherein the signal is detected using a detection apparatus comprising one selected from the group consisting of a microscope and a photomultiplier tube.

35. The method of claim 34, wherein a processor coupled to the detection apparatus determines the quantity of the target nucleic acids that was present in the sample

36. The method of claim 35, wherein the process determines a copy number for the locus in the target nucleic acid.

37. The method of claim 22, wherein the step are performed as a prenatal test for aneuploidy in a fetus.
